(19) United States
(12) Patent Application Publication
(10) Pub. No.: US 2006/0088590 A1
(43) Pub. Date: Apr. 27, 2006

(54) Non-blooming gelatin and non-gelatin formulations

(75) Inventors: Karunakar Sukuru, High Point, NC (US); Nachiappan Chidambaram, High Point, NC (US); Aqeel A. Fatmi, Greensboro, NC (US)

Correspondence Address: PATREA L. PABST;PABST PATENT GROUP LLP, 400 COLONY SQUARE, SUITE 1200, ATLANTA, GA, 30361, US

(73) Assignee: Banner Pharmacaps, Inc.

(21) Appl. No.: 11/083,869

(22) Filed: Mar. 18, 2005

Related U.S. Application Data
(60) Provisional application No. 60/621,260, filed on Oct. 22, 2004.

(51) Int. Cl. A61K 9/64

(52) U.S. Cl. 424/456

(57) ABSTRACT

A non-blooming plasticizer composition and methods of using the composition are described herein. The composition is a mixture of sorbitol and sorbitan wherein the ratio of sorbitan and sorbitol is between about 0.40 and about 1.2 by weight, more preferably from about 0.45-0.50 to about 0.70 by weight, wherein the composition contains less than 20% of other dextrose hydrolytic degradation products. The non-blooming plasticizer compositions can be mixed, alone or in combination with other shell additives, with gelatin or non-gelatin materials to prepare soft capsules for the delivery of pharmaceutical, nutritional and personal care products, such as bath oils, shampoos and conditioners, and skin lotions.

### CROSS-REFERENCE TO RELATED APPLICATIONS

This application claims priority to U.S. Ser. No. 60/621,260, entitled “Non-Blooming Gelatin Formulations”, to Karunakar Sukuru, Nachiappan Chidambaram And Aqeel A. Fatmi, filed Oct. 22, 2004.

### FIELD OF THE INVENTION

The invention is in the field of soft gelatin and non-gelatin capsules.

### BACKGROUND OF THE INVENTION

Soft capsules are commonly used as a dosage form for administering pharmaceutical, nutritional and personal care products. Soft capsules have several advantages over other dosage forms including enhanced dissolution rates, enhanced bioavailability of active agents with poor water solubility, protection of active agents susceptible to oxidation, ability to mask unpleasant odors and/or tastes of active agents, and increased flexibility in selecting size, shape, and color in order to identify and differentiate products.

Soft capsules are composed of a capsule content (“fill”) encapsulated in a soft gelatin or non-gelatin shell. Non-gelatin materials include carbohydrates such as carrageenan and starches. For soft capsules manufactured using a rotary die encapsulation process, the fill is typically a liquid or a combination of miscible liquids, a solution of a solid(s) in a liquid(s), or a suspension of solid(s) in a liquid. Polyethylene glycol (PEG) is commonly included in the fill as a solvent for the active agent to be encapsulated. The capsule shell is composed primarily of gelatin or non-gelatin materials, a plasticizer, and purified water.

Glycerin has been used as an effective plasticizer for soft capsules containing hydrophilic fills due to the strong intermolecular interactions between the hydroxy groups of glycerin and the hydrophilic groups on gelatin or non-gelatin materials. However, glycerin-plasticized soft capsules with hydrophilic PEG-based fill compositions become brittle over time as both glycerin and water migrate from the shell to the hygroscopic fill. To prevent embrittlement, glycerin has been used in combination with non-crystallizing sorbitol solutions to plasticize soft capsules with hydrophilic formulations. Sorbitol is less hydrophilic, has a higher molecular weight, and is less soluble in PEG than glycerin, resulting in a lower tendency to migrate into the PEG-based fills.

Blooming occurs when crystalline sorbitol in the shell recrystallizes, forming white precipitates on the surface of the capsule. While blooming poses little danger to the consumer, these white precipitates can obscure the printing on the capsule surface making identification of the product more difficult and may result in a product being recalled from the market.

It is therefore an object of the invention to provide plasticizer compositions containing sorbitol and sorbitan, which are not susceptible to blooming.

It is still another object of the invention to provide improved soft capsules dosage unit forms for the delivery of pharmaceutically active agents.

### BRIEF SUMMARY OF THE INVENTION

A non-blooming plasticizer composition and methods of using the composition are described herein. The composition is a mixture of sorbitol and sorbitan wherein the ratio of sorbitan and sorbitol is between about 0.40 and about 1.2 by weight, more preferably from about 0.50 to about 0.80 by weight and wherein the compositions contains less than 20% by weight of other dextrose hydrolytic degradation products. The non-blooming plasticizer compositions, alone or in combination with other shell additives including other plasticizers such as glycerin, propylene glycol and maltitol, can be mixed with gelatin or non-gelatin materials to prepare soft capsules for the delivery of pharmaceutical, nutritional and personal care products, such as bath oils, shampoos and conditioners, and skin lotions.

### DETAILED DESCRIPTION OF THE INVENTION

I. Soft Capsule Shell

A. Gelatin

Gelatin is the product of the partial hydrolysis of collagen. Gelatin is classified as either Type A or Type B gelatin. Type A gelatin is derived from the acid hydrolysis of collagen while Type B gelatin is derived from alkaline hydrolysis of collagen. Traditionally, bovine bones and skins have been used as raw materials for manufacturing Type A and Type B gelatin while porcine skins have been used extensively for manufacturing Type A gelatin. In general acid-processed gelatins form stronger gels than lime-processed gelatins of the same average molecular weight.

B. Non-Gelatin Materials

Carrageenan is a collective term for polysaccharides prepared by alkaline extraction (and modification) from red seaweed (Rhodophycae), mostly of genus Chondrus, Eucheuma, Gigartina and Iridaea. Different seaweeds produce different carrageenans. Carrageenan consists of alternating 3-linked-β-D-galactopyranose and 4-linked-α-D-galactopyranose units. Most, if not all, of the galactose units are substituted with sulfate ester groups.

C. Non-Blooming Plasticizer

Plasticizers are chemical agents added to gelatin to make the material softer and more flexible. Suitable plasticizers include glycerin, sorbitol solutions which are mixtures of sorbitol and sorbitan, and other polyhydric alcohols such as propylene glycol and maltitol or combinations thereof.

As used herein, “sorbitol” generally refers to six carbon polyols (sugar alcohols). The structures of linear and cyclic sorbitol are shown below.

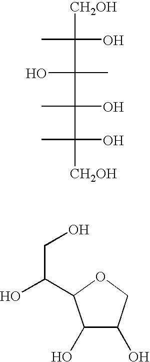

As used herein, “sorbitan” generally refers to a mixture of mono-, di-, and tri-sorbitol anhydrides. Suitable sorbitans include 1,4-sorbitan, isosorbide, and other hexitans and related isomers and combinations thereof.

As used herein, “other dextrose hydrolytic degradation products” generally refers to other polyols which are suitably hydrogenated saccharides. Such polyols are produced by the partial or complete hydrolysis of glucose syrup (dextrose).

A commercially available sorbitol solution is Sorbitol Special, manufactured by SPI Pharma, Inc. Sorbitol Special contains 40-55% sorbitol, 15-30% sorbitan, and 1-10% mannitol. It has a total solids content of 76% and water content of 24%. However, Sorbitol Special has been known to exhibit blooming in certain applications. U.S. Pat. No. 4,780,316 to Brox describes a softgel wherein the plasticizer contains by weight 25-45% D-sorbitol, 20-30% sorbitan (the major component being 1,4-sorbitan), 1-6% mannitol and 20-25% other polyols which are suitable hydrogenated saccharides. Brox discloses that these polyols are derived from the hydrolysis and partial hydrolysis of glucose syrup.

In a preferred embodiment, the plasticizer is a mixture of sorbitol and sorbitan wherein the sorbitan to sorbitol ratio is between about 0.40 and about 1.2, more preferably between about 0.50 and about 0.80 by weight. Suitable sorbitans include 1,4-sorbitol anhydride, 2,5-sorbitol anhydride, isosorbide, and combinations thereof. The plasticizer may contain other polyols which are suitably hydrogenated saccharides produced by the hydrolytic degradation of dextrose provided the concentration of these polyols is less than 20% by weight. The plasticizer is prepared by reacting cyclic sorbitol with a nickel catalyst at high temperatures. The ratio of sorbitan to sorbitol is monitored during the reaction and the reaction is terminated when the desired ratio is reached. Cyclic sorbitol can be prepared by the dehydration of linear sorbitol using, for example, p-toluenesulfonic acid. Sorbitol is produced by the hydrogenation of glucose. The plasticizer may also contain less than 20% by weight of other hydrolytic degradation products of dextrose.

The ratio, by weight, of dry plasticizer to dry gelatin or non-gelatin material determines the “hardness” of the capsule shell and is from about 0.4:1 to about 0.8:1. The exact ratio is selected based on the fill formulation and the anticipated storage conditions of the marketed product. Hydrophilic fills require greater plasticizer to gelatin ratios than lipophilic fills to compensate for any plasticizer migration into the fill over time.

Capsule size may also influence the selection of plasticizer to gelatin ratio. For the same fill composition, orally administered capsules larger than 10 minims (1 minim= 1/60 fluid dram=⅛ fluid ounce) in size typically have a higher content of plasticizer for increased ease of swallowing. Lower concentrations of plasticizer are recommended for oxygen-labile active agents since oxygen permeability of gelatin and non-gelatin films increases with increasing amounts of plasticizer. The capsule shell can be “overplasticized” in order to prepare chewable soft capsules as described in U.S. Pat. No. 6,258,380 to Overholt.

D. Other Shell Additives

In addition to the plasticizer(s), other suitable shell additives include opacifiers, colorants, humectants, preservatives, flavorings, and buffering salts and acids.

Opacifiers are used to opacify the capsule shell when the encapsulated active agents are light sensitive. Suitable opacifiers include titanium dioxide, zinc oxide, calcium carbonate and combinations thereof.

Colorants can be used to for marketing and product identification/differentiation purposes. Suitable colorants include synthetic and natural dyes and combinations thereof.

Humectants can be used to suppress the water activity of the soft capsule. Suitable humectants include glycerin and sorbitol, which are often components of the plasticizer composition. Due to the low water activity of dried, properly stored soft capsules, the greatest risk from microorganisms comes from molds and yeasts. For this reason, preservatives can be incorporated into the capsule shell. Suitable preservatives include alkyl esters of p-hydroxy benzoic acid such as methyl, ethyl, propyl, butyl and heptyl (collectively known as “parabens”) or combinations thereof.

Flavorings can be used to mask unpleasant odors and tastes of fill formulations. Suitable flavorings include synthetic and natural flavorings. The use of flavorings can be problematic due to the presence of aldehydes which can cross-link gelatin. As a result, buffering salts and acids can be used in conjunction with flavorings that contain aldehydes in order to inhibit cross-linking of the gelatin.

II. Fill Material

A. Agents

Soft capsules can used to deliver a wide variety of pharmaceutically active agents. Exemplary agents include analgesics, anti-inflammatory agents, antihelmintics, anti-arrhythmic agents, anti-bacterial agents, anti-viral agents, anti-hypertensive agents, anti-coagulants, anti-depressants, anti-diabetics, anti-epileptics, anti-fungal agents, anti-gout agents, anti-malarials, anti-migraine agents, anti-muscarinic agents, anti-neoplastic agents, erectile dysfunction improvement agents, immunosupressants, anti-protozoal agents, anti-thyroid agents, anxiolytic agents, sedatives, hypnotics, neuroleptics, β-blockers, cardiac inotropic agents, corticosteroids, diuretics, anti-parkinsonian agents, gastro-intestinal agents, histamine H₁ and H₂ receptor antagonists, keratolytics, lipid regulating agents, anti-anginal agents, nutritional agents, opioid analgesics, sex hormones, stimulants, muscle relaxants, anti-osteoporosis agents, anti-obesity agents, cognition enhancers, anti-urinary incontinence agents, nutritional oils, anti-benign prostate hypertrophy agents, essential fatty acids, non essential fatty acids, vitamins, minerals and mixtures thereof.

B. Excipients

Formulations may be prepared using a pharmaceutically acceptable carrier composed of materials that are considered safe and effective and may be administered to an individual without causing undesirable biological side effects or unwanted interactions. The carrier is all components present in the pharmaceutical formulation other than the active ingredient or ingredients. As generally used herein “carrier” includes, but is not limited to, polyethylene glycols, surfactants, humectants, vegetable oils, medium chain mono, di and triglycerides, lecithin, waxes, hydrogenated vegetable oils, colloidal silicon dioxide, povidone, celluloses, carbopol, acrylate polymers, other hydrogel forming polymers, plasticizers, crystallization inhibitors, wetting agents, bulk filling agents, solubilizers, bioavailability enhancers and combinations thereof.

In a preferred embodiment, polyethylene glycol is used a solubilizer and is present in a concentration from about 5 to about 10%. The molecular weight of propylene glycol is between 300 and 600.

III. Method of Making

The main ingredients of the softgel capsule shell are gelatin, plasticizer, and purified water. Typical gel formulations contain (w/w) 40-50% gelatin, 20-30% plasticizer, and 30-40% purified water. Most of the water is subsequently lost during capsule drying. The ingredients are combined to form a molten gelatin mass using either a cold melt or a hot melt process. The prepared gel masses are transferred to preheated, temperature-controlled, jacketed holding tanks where the gel mass is aged at 50-60° C. until used for encapsulation.

A. Cold Melt Process

The cold melt process involves mixing gelatin with plasticizer and chilled water and then transferring the mixture to a jacket-heated tank. Typically, gelatin is added to the plasticizer at ambient temperature (18-22° C.). The mixture is cooked (57-95° C.) under vacuum for 15-30 minutes to a homogeneous, deaerated gel mass. Additional shell additives can be added to the gel mass at any point during the gel manufacturing process or they may be incorporated into the finished gel mass using a high torque mixer.

B. Hot Melt Process

The hot melt process involves adding, under mild agitation, the gelatin to a preheated (60-80° C.) mixture of plasticizer and water and stirring the blend until complete melting is achieved. While the hot melt process is faster than the cold melt process, it is less accurately controlled and more susceptible to foaming and dusting.

C. Soft Capsules

Soft capsules are typically produced using a rotary die encapsulation process. The gel mass is fed either by gravity or through positive displacement pumping to two heated (48-65° C.) metering devices. The metering devices control the flow of gel into cooled (10-18° C.), rotating casting drums. Ribbons are formed as the cast gel masses set on contact with the surface of the drums.

The ribbons are fed through a series of guide rolls and between injection wedges and the capsule-forming dies. A food-grade lubricant oil is applied onto the ribbons to reduce their tackiness and facilitate their transfer. Suitable lubricants include mineral oil, medium chain triglycerides, and soybean oil. Fill formulations are fed into the encapsulation machine by gravity. In the preferred embodiment, the soft capsules contain printing on the surface, optionally identifying the encapsulated agent and/or dosage.

IV. Method of Use

The soft capsules may encapsulate a wide range of pharmaceutically active agents, nutritional agents and personal care products. Soft capsules may be administered orally to a patient to deliver a pharmaceutically active agent.

While orally administered soft capsules are intended to provide immediate release of the encapsulated contents, soft capsules can be modified for controlled, delayed or enteric release.

The present invention will be further understood by reference to the following non-limiting examples. Examples 1-8 describe exemplary fill formulations; Examples 9-15 describe soft gel formulation, with examples 9 and 10 being examples of formulations that bloom.

### EXAMPLES

### Example 1

| Fill ingredients | % by wt. |
|---|---|
| Ibuprofen | 37 |
| PEG600 | 37 |
| Kollidon | 20 |
| Vit. E TPGS* | 6 |

*Vit E TPGS is tocophenol glycol succinate

### Example 2

| Fill ingredients | % by wt. |
|---|---|
| Acetaminophen | 50 |
| Polyethylene glycol | 42 |
| Propylene glycol | 5 |
| Kollidon | 2 |
| Colloidal silicondioixde | 1 |

### Example 3

| Fill ingredients | % by wt. |
|---|---|
| Acetaminophen | 50 |
| Polyethylene glycol | 42 |
| Propylene glycol | 5 |
| Kollidon | 2 |
| Colloidal silicondioixde | 1 |

### Example 4

| Fill ingredients | % by wt. |
|---|---|
| Acetaminophen | 50 |
| Dextromethorphan | 1.5 |
| hydrobromide | |
| Pseudoephedrine hydrochloride | 3 |
| Doxylamine succinate | 0.6 |
| Polyethylene glycol | 36.9 |
| Propylene glycol | 5 |
| Kollidon | 2 |
| Colloidal silicondioixde | 1 |

### Example 5

| Fill ingredients | % by wt. |
|---|---|
| Dextromethorphan | 7.5 |
| hydrobromide | |
| Polyethylene glycol | 82.5 |
| Propylene glycol | 5 |
| Polyvinyl pyrrolidone | 5 |

### Example 6

| Fill ingredients | % by wt. |
|---|---|
| Naproxen sodium | 25.5 |
| Acetic acid | 3 |
| Polyethylene glycol | 62.30 |
| Water, purified | 7.4 |
| Polyvinyl pyrrolidone | 1.8 |

### Example 7

| Fill ingredients | % by wt. |
|---|---|
| Vit. E | 100 |

### Example 8

| Fill ingredients | % by wt. |
|---|---|
| Vit. E | 10 |
| Vit. C | 50 |
| Beta carotene | 10 |
| Vit. D | 0.1 |
| Vit. B1 | 1 |
| Vit B6 | 1 |
| Soya lecithin | 3 |
| Bees wax | 3 |
| Hydrogenated veg. Oil | 3 |
| Vegetable oil | 18.9 |

Shell/Sheath Composition:

### Example 9

| Shell ingredients | % by wt. |
|---|---|
| Gelatin | 42 |
| Sorbitan, sorbitol (ratio 0.2) | 24 |
| Water, purified | 34 |

### Example 10

| Shell ingredients | % by wt. |
|---|---|
| Gelatin | 42 |
| Sorbitan, sorbitol (ratio 0.35) | 24 |
| Water, purified | 34 |

### Example 11

| Shell ingredients | % by wt. |
|---|---|
| Gelatin | 42 |
| Sorbitan, sorbitol (ratio 0.4) | 24 |
| Water, purified | 34 |

### Example 12

| Shell ingredients | % by wt. |
|---|---|
| Gelatin | 42 |
| Sorbitan, sorbitol (ratio 0.45) | 24 |
| Water, purified | 34 |

### Example 13

| Shell ingredients | % by wt. |
|---|---|
| Gelatin | 42 |
| Sorbitan, sorbitol (ratio 0.62) | 24 |
| Water, purified | 34 |

### Example 14

| Shell ingredients | % by wt. |
|---|---|
| Gelatin | 42 |
| Sorbitan, sorbitol (ratio 0.45) | 12 |
| Glycerin | 10 |
| Water, purified | 34 |

### Example 15

| Shell ingredients | % by wt. |
|---|---|
| Carrageenan | 8 |
| Modified starch | 27.1 |
| Sorbitan, sorbitol (ratio 0.62) | 25.4 |
| Water, purified | 39.5 |

Those skilled in the art will recognize, or be able to ascertain using no more than routine experimentation, many equivalents to the specific embodiments of the invention described herein. Such equivalents are intended to be encompassed by the following claims.

We claim:

1. A non-blooming gelatin and non-gelatin plasticizer composition comprising a mixture of sorbitol and sorbitan wherein the ratio of sorbitan to sorbitol is between about 0.40 and about 1.2 by weight and wherein the composition comprises less than 20% of other dextrose hydrolytic degradation products.

2. The composition of claim 1, wherein the ratio of sorbitan to sorbitol is 0.50 to 0.70 by weight.

3. The composition of claim 1 wherein the composition comprises other plasticizers selected from the group consisting of glycerin, propylene glycol or maltitol.

4. A method of making a non-blooming soft capsule shell comprising providing in the soft capsule shell composition a plasticizer comprising a mixture of sorbitol and sorbitan wherein the ratio of sorbitan to sorbitol is between about 0.40 and about 1.2 by weight and wherein the composition comprises less than 20% of other dextrose hydrolytic degradation products.

5. The method of claim 4 further comprising encapsulating a fill material in the soft capsule shell.

6. The method of claim 4 wherein the soft capsule shell composition comprises the plasticizer and gelatin.

7. The method of claim 4, wherein the ratio of sorbitan to sorbitol is between about 0.50 and 0.70 by weight.

8. A soft gel capsule for making a pharmaceutical dosage form comprising a non-blooming plasticizer composition comprising a mixture of sorbitol and sorbitan wherein the ratio of sorbitan to sorbitol is between about 0.40 and about 1.2 by weight and wherein the composition comprises less than 20% of other dextrose hydrolytic degradation products.

9. The soft gel capsule of claim 8, wherein the ratio of sorbitan to sorbitol is 0.50 to 0.70 by weight.

10. The soft gel capsule of claim 8 wherein the soft capsule shell further comprises other plasticizers selected from the group consisting of glycerin, propylene glycol or maltitol.

11. The soft gel capsule of claim 8 wherein the soft capsule shell comprises gelatin.

12. The soft gel capsule of claim 8 comprising at least one pharmaceutically active agent in a therapeutically effective amount encapsulated in the soft capsule shell.

13. The soft gel capsule of claim 12 wherein the agent is selected from the group consisting of analgesics, anti-inflammatory agents, antihelmintics, anti-arrhythmic agents, anti-bacterial agents, anti-viral agents, anti-hypertensive agents, anti-coagulants, anti-depressants, anti-diabetics, anti-epileptics, anti-fungal agents, anti-gout agents, anti-malarials, anti-migraine agents, anti -muscarinic agents, anti-neoplastic agents, erectile dysfunction improvement agents, immunosupressants, anti-protozoal agents, anti-thyroid agents, anxiolytic agents, sedatives, hypnotics, neuroleptics, β-blockers, cardiac inotropic agents, corticosteroids, diuretics, anti-parkinsonian agents, gastro-intestinal agents, histamine H₁ and H₂ receptor antagonists, keratolytics, lipid regulating agents, anti-anginal agents, nutritional agents, opioid analgesics, sex hormones, stimulants, muscle relaxants, anti-osteoporosis agents, anti-obesity agents, cognition enhancers, anti-urinary incontinence agents, nutritional oils, anti-benign prostate hypertrophy agents, essential fatty acids, non essential fatty acids, vitamins, minerals and mixtures thereof.
